(19) United States
(12) Patent Application Publication
(10) Pub. No.: US 2008/0152715 A1
(43) Pub. Date: Jun. 26, 2008

(54) Method for preparing nanoparticles comprising vitamin K and poly-isopropyl-butyl methacrylate-acrylic acid copolymer with supercritical fluid using molecular association theory

(75) Inventors: Moon Sam Shin, Seoul (KR); Yongjin Lee, Inchon (KR); Hwayong Kim, Seoul (KR)

Correspondence Address: GREER, BURNS & CRAIN, 300 S WACKER DR, 25TH FLOOR, CHICAGO, IL, 60606, US

(73) Assignee: Seoul National Universtiy Industry Foundation

(21) Appl. No.: 11/654,761

(22) Filed: Jan. 17, 2007

(30) Foreign Application Priority Data
Dec. 26, 2006 (KR) 10-2006-0134131

(51) Int. Cl. A61K 9/14

(52) U.S. Cl. 424/489; 424/280.1; 977/906

(57) ABSTRACT

The present invention relates to a method for preparing supercritical nanoparticles of vitamin K by a supercritical fluid process.

The basic concept of the preparation method is as follows. A nanoscaled vitamin K complex is prepared by dissolving vitamin K and a biodegradable polymer in a suitable amount of an organic solvent, spraying the solution into a reactor equilibrated with a supercritical carbon dioxide via a nozzle to obtain particles, introducing supercritical carbon dioxide several times to the particles, extracting the organic solvent out, and then removing carbon dioxide.

### BACKGROUND OF THE INVENTION

1. Field of the Invention

The present invention relates to a method for preparing supercritical nanoparticles of vitamin K by a supercritical fluid process using a molecular association theory about density fluctuations near a critical point, and specifically to a method comprising the steps of spraying a solution obtained by dissolving vitamin K as a drug and a biodegradable polymer in an organic solvent into a supercritical fluid to prepare nanoparticles of vitamin K and the biodegradable polymer; and introducing a fresh supercritical fluid to the nanoparticles to remove the organic solvent.

2. Description of the Related Art

There are many factors which cause skin diseases such as stretch marks, but satisfactory and safe therapeutic drugs have not yet been developed. Many therapeutic agents have been used to treat these diseases. However, there exist many problems in treatment of skin diseases, such as improper solubility in water or organic solvents, limitation of absorption through the skin, and the side effect due to the residual solvent by using an organic solvent, upon preparing fine particles or nanoparticles.

There have been made attempts to use vitamin K as a therapeutic agent for skin diseases. However, in practice, the side effects by the residual organic solvent still give the limitation in using the vitamin K as a therapeutic agent for skin diseases. In order to solve the problems caused from the residual organic solvent, the present invention aims to use a supercritical fluid as a substitute for the organic solvents. Any study for preparation of fine particles comprising vitamin K with a supercritical fluid has not yet been made. Hereinbelow, the supercritical fluid will be described in detail.

A supercritical fluid is an incompressible fluid under a temperature and pressure above each of the critical points, which shows unique characteristics not shown in the conventional organic solvents. Specifically, since the supercritical fluid near a critical point has molecular association by a density fluctuation, it has excellent properties such as a high density close to that of a liquid, a low viscosity and a high diffusion coefficient close to those of a gas, respectively, and a very low surface tension, simultaneously. Since the density of a supercritical fluid can be continuously changed from a sparse state like an ideal gas to a highly dense state like a liquid, its physical properties at equilibrium (e.g., a solubility, and an entrainer effect), mass transfer properties (e.g., a viscosity, a diffusion coefficient and a thermal conductivity) and a molecular clustering state of the fluid can be regulated.

Therefore, by using easy regulation of the properties of the supercritical fluid, it is possible to attain solvent characteristics, which correspond to a combination of those of several solvents. Carbon dioxide, in particular, has a critical temperature of 304.2 K, similar to room temperature, so that it is suitable for use in a thermally unstable substance such as a drug. Further, since the carbon dioxide has many advantages that it is nontoxic, incombustible, inexpensive and recyclable for reuse, as well as it can be employed in an environment-friendly process, it is very ideal for application in medical products.

Many studies to take advantages of such the unique properties of a supercritical fluid have recently been made in various fields, such as selective extraction of a target material and analysis of materials through extraction. Further, recrystallization or methods for obtaining microparticles using the supercritical fluid as a solvent or an anti-solvent have been actively studied.

In addition, in order to improve the skin permeability, a therapeutic agent should have a particle size of 50 to 100 nanometers. A drug with a particle size more than 100 nm may cause reduction of the skin permeability, whereas the drug with a particle size less than 50 nm may permeate to dermis, even to increase the toxicity. Furthermore, the drugs prepared by the conventional supercritical process have a problem of poor skin permeability due to its particle size of 1 to 10 micron.

### SUMMARY OF THE INVENTION

It is an object of the present invention to solve the above-described problems, and to provide an optimal method for preparing a therapeutic agent for skin diseases, which involves employing vitamin K, which had been never produced or studied by a supercritical process, in the supercritical process to solve the problem due to a residual organic solvent; and comprises nanoscaling vitamin K having a large particle size to enhance skin absorption; and incorporating a biodegradable polymer in combination with a drug to control the efficiency release rate of the drug, whereby the sustained release of vitamin K can be enhanced.

### DESCRIPTION OF THE PREFERRED EMBODIMENTS

Hereinafter, the present invention will be described in detail.

The basic concept of the preparation method is as follows. A nanoscaled vitamin K complex is prepared by dissolving vitamin K and a biodegradable polymer in a suitable amount of an organic solvent, spraying the solution into a reactor equilibrated with a supercritical carbon dioxide via a nozzle to obtain particles, introducing supercritical carbon dioxide several times to the particles, extracting the organic solvent out, and then removing carbon dioxide.

More specifically, the method comprises the steps of dissolving vitamin K and a biodegradable polymer in an organic solvent to prepare a solution; spraying a solution into the reactor charged with a supercritical fluid to prepare spherical vitamin K nanoparticles; introducing a fresh supercritical fluid into the reactor containing the spherical vitamin K nanoparticles to remove the organic solvent; and recovering spherical vitamin K nanoparticles after removal of the organic solvent.

Hereinbelow, each of the steps for the preparation method will be described in detail.

Vitamin K is useful as an adjuvant for treatment of skin diseases such as stretch marks, but has been limited in its use due to the side effects such as a problem of the residual solvent. Accordingly, in order to overcome the side effects due to the residual solvent, a supercritical fluid has been used, wherein it is critical to maintain the spherical particles with a maximum surface area.

(1) Step of Preparation of Solution

In the invention, vitamin K is used in a weight proportion of 0.0015 to 0.0018 wt % of total materials (vitamin K, biodegradable polymer, supercritical fluid and solvent), since if the proportion of vitamin K is less than 0.0015 wt %, the spherical shape of vitamin K nanoparticles can not be obtained, and if that of vitamin K is more than 0.0018 wt %, the nanoscaled particles of spherical vitamin K can not be obtained.

A biodegradable polymer is used for regulating the drug release rate, and particularly preferred is a poly-isopropyl-butyl methacrylate-acrylic acid copolymer. A poly-isopropyl-butyl methacrylate-acrylic acid copolymer has both of acrylic acid as a hydrophilic part and butyl methacrylate as a hydrophobic part in a molecule, which prevents particle aggregation generated upon nucleation and nuclear growth of the drug, that is, functions as a surfactant. Therefore, the drug in the form of the nanoparticles with a smaller size can be obtained. The proportion of a poly-isopropyl-butyl methacrylate-acrylic acid copolymer is preferably 0.0013 to 0.0018 wt % of total materials. In the case where the proportion of the biodegradable polymer is less than 0.0013 wt % or more than 0.0015 wt %, a vitamin K-biodegradable polymer complex can not be obtained in the sphere shape. Another reason for using a poly-isopropyl-butyl methacrylate-acrylic acid copolymer as a biodegradable polymer is that spherical particles cannot be formed with other biodegradable polymers.

As an organic solvent, methanol, ethanol, or the like is used, but in the invention, ethanol having a low toxicity to a human is used.

(2) Step of Formation of Spherical Structure

As the supercritical fluid, supercritical carbon dioxide, supercritical dinitrogen monoxide, supercritical trifluoromethane, supercritical propane, supercritical ethylene, supercritical xenon, or the like is employable, but in a preferred example of the invention, supercritical carbon dioxide is used.

Carbon dioxide is injected into a stainless steel reactor, until the temperature and the pressure in the reactor each reach above 304.2 K and 73.8 bar (which correspond to the critical temperature and the critical pressure of carbon dioxide, respectively). The reactor is maintained at a supercritical state with applying the pressure and elevating the temperature, and left to be equilibrated in a supercritical state. To maintain a constant pressure upon applying the pressure to carbon dioxide and to check the exact amount of the fluid injected, a syringe pump is used and to maintain the constant temperature, a circular thermostat or an automatic temperature regulator is preferably used.

When the inside of the reactor reaches an equilibrated supercritical state, the solution of vitamin K and the additives prepared in Step (1) is injected into the reactor at a constant rate using a small liquid pump capable of speed control. At this time, in order to prevent the nozzle from clogging, it is preferable to inject a small amount, for example, 3 to 4 ml, of a blank solvent, prior to injecting the solution. As the amount of the blank solvent injected increases, the time required for washing the reactor with the supercritical fluid becomes longer.

The solution injected is sprayed via a nozzle. The organic solvent in the sprayed solution is blended with the supercritical carbon dioxide rapidly, to form particles. In order to prevent the supercritical fluid in the reactor from being saturated during the solution injection, a fresh supercritical fluid may further be injected into the reactor.

(3) Step of Removal of Organic Solvent

After the solution is completely sprayed into the reactor, a particle washing step is required by introducing a fresh supercritical fluid into the reactor to remove the organic solvent from the formed particles. In the above step, the supercritical fluid is injected at the constant rate, and discharged via an outlet at the same rate as the injection rate, which maintains the reactor pressure at 150 bars being allowed to obtain a particle size of 100 nm or less. At this time, a back pressure regulator is connected to the outlet to maintain the constant pressure in the reactor by regulating the discharging rate.

Double overlapping membrane filters having a pore size of 0.45 μm are installed at the outlet to hold the particles within the reactor. The washing step should be repeated until the residual solvent is completely removed, since if there is the residual solvent, the solvent re-dissolves the particles formed by precipitation, thus generating coagulation upon decreasing the temperature and the pressure for the recovery of the particles. The amount of the supercritical fluid for washing varies depending on the amount of the organic solvent used and the reactor size, preferably about 2,000 to 3,000 ml.

4) Step of Recovery of Particles

When the washing step is completed, the supply of the supercritical fluid into the reactor is stopped and the supercritical fluid is discharged. At this time, if the supercritical fluid is too rapidly discharged, the particles may be damaged. Accordingly, it is preferable to slowly discharge the supercritical fluid. After the supercritical fluid in the reactor is completely removed, the particles are recovered from the wall or bottom of the reactor.

Hereinafter, the present invention will be described in detail with reference to Examples.

The pretreated vitamin K has a particle size of 50 to 100 microns. In Examples, the particle sizes of vitamin K nanoparticles prepared by the following conditions were measured using a Particle Size Analyzer. Here, spherical fine particles having a particle size of 50 to 300 nm can be obtained according to the presence or absence of a poly-isopropyl-butyl methacrylate-acrylic acid copolymer as a biodegradable polymer, and the operating conditions.

As shown in the following Table 1, the present Examples represent the operating conditions of the reactor in the step of formation of a spherical structure. The temperature and the pressure of the reactor must be each above 304.2 K and 73.8 bar, which correspond to the critical temperature and the critical pressure of carbon dioxide, respectively. The operating parameters in the reactor are the temperature and the pressure in reactor, the amount of carbon dioxide as a supercritical fluid, and the amount of solution prepared in the preparation step. In Example 1, the solution contains vitamin K of 0.0016 wt % alone without a biodegradable polymer. In Example 2 to 8, the solution contains both of vitamin K of 0.0016 wt % and the poly-isopropyl-butyl methacrylate-acrylic acid copolymer of 0.0014 wt % as a biodegradable polymer. It is noted that the particle sizes are decreased to around 100 nm in the case of the solution containing the poly-isopropyl-butyl methacrylate-acrylic acid copolymer as a biodegradable polymer. In Examples 1 and 3, the change was measured at 313K and 323K by adjusting the reactor temperature. As a result, if the temperature is increased, the particle size tends to be increased. In Examples 1, 4 and 5, if the reactor pressure was adjusted from 130 to 170 bars, particles with the optimal sizes can be obtained at a reactor pressure of 150 bars. In Examples 1, 6 and 7, if the flow rate of carbon dioxide was changed from 2.5 to 3.0 kg/hr, particles with the optimal sizes can be obtained in the flow rate of carbon dioxide of 2.5 to 2.7 kg/hr. In Examples 1 and 8, the flow rate of the solution was changed from 0.5 to 1.0 ml/min, and it was found that as the amount of the solution is increased, the particle size is increased.

TABLE 1
Operating conditions and particle sizes
according to four operating parameters in Example 1 to 8

| | | | Flow rate of | | |
|---|---|---|---|---|---|
| | Reactor | Reactor | carbon | Flow rate of | particle |
| | temperature | pressure | dioxide | solution | size |
| Example | (K) | (bar) | (kg/hr) | (ml/min) | (nm) |
| Example 1 | 313 | 150 | 2.5 | 0.5 | 589 |
| Example 2 | 313 | 150 | 2.5 | 0.5 | 86 |
| Example 3 | 323 | 150 | 2.5 | 0.5 | 135 |
| Example 4 | 313 | 130 | 2.5 | 0.5 | 120 |
| Example 5 | 313 | 170 | 2.5 | 0.5 | 103 |
| Example 6 | 313 | 150 | 2.7 | 0.5 | 73 |
| Example 7 | 313 | 150 | 3.0 | 0.5 | 149 |
| Example 8 | 313 | 150 | 2.5 | 1.0 | 200 |

The operating conditions of Example 2 and 6 are suitable for obtaining preferable nanoparticles of 50 to 100 nm. That is, the particle size could be significantly decreased by incorporating the poly-isopropyl-butyl methacrylate-acrylic acid copolymer as a biodegradable polymer. The optimal condition is such that the temperature is 313 K, the pressure is 150 bars, the flow rate of carbon dioxide is 2.5 to 2.7 kg/hr and the flow rate of the solution is 0.5 ml/min.

From more specifically performed experiments, it was found that in order to obtain supercritical nanoparticles having a particle size of 50 to 100 nm, the proportions of vitamin K and the poly-isopropyl-butyl methacrylate-acrylic acid copolymer are 0.0016 wt % and 0.0014 wt %, respectively. It was also found that nanoparticles with a desired range of the preferable sizes can be obtained, if the operating conditions are that the temperature is 313.0 to 313.4 K, the pressure is 149.5 to 151.3 bars, the flow rate of carbon dioxide is 2.5 to 2.7 kg/hr, and the flow rate of the solution is 0.47 to 0.50 ml/min. Under the operating conditions below the temperature and above the pressure and the flow rate, the nanoscaled particles of spherical vitamin K can not be obtained. On operating conditions above the temperature, the vitamin K is apt to degradable and under the operating conditions below the pressure and the flow rate, unwanted nanoparticles with a particle size more than 100 nm or more are obtained, thus it not being preferable.

According to the present invention, nanoparticles of vitamin K can be prepared by using a supercritical fluid process which has not been conventionally performed, a drug release rate can be regulated by incorporating a poly-isopropyl-butyl methacrylate-acrylic acid copolymer as a biodegradable polymer, and supercritical nanoparticles having a particle size of 50 to 100 nm (Examples 2 and 6) can be obtained by controlling the conditions of the process, whereby the effective cross-sectional area is increased to improve skin permeability.

What is claimed is:

1. A method for preparing a therapeutic agent for skin diseases in the form of nanoparticles comprising vitamin K and a poly-isopropyl-butyl methacrylate-acrylic acid copolymer with a supercritical fluid using a molecular association theory, wherein
the method comprises the steps of:
dissolving vitamin K and a biodegradable polymer in an organic solvent to prepare a solution (step 1), the proportion of vitamin K is 0.0015 to 0.0018 wt %, the biodegradable polymer is the poly-isopropyl-butyl methacrylate-acrylic acid copolymer, the proportion of the poly-isopropyl-butyl methacrylate-acrylic acid copolymer is 0.0013 to 0.0015 wt %, and ethanol is used as an organic solvent;
spraying the solution into a reactor charged with a supercritical fluid to prepare spherical vitamin K nanoparticles (step 2), carbon dioxide is used as a supercritical fluid, the temperature and the pressure in the reactor are 313.0 to 313.4 K, and 149.5 to 151.3 bars, respectively, the flow rates of carbon dioxide and the solution are 2.5 to 2.7 kg/hr, and 0.47 to 0.50 ml/min, respectively;
introducing a fresh supercritical fluid into the reactor containing the spherical vitamin K nanoparticles to remove the organic solvent (step 3), a back pressure regulator is connected to the outlet to maintain the constant pressure in the reactor by regulating the pressure of the supercritical fluid introduced into the reactor, and the pressure at the outlet is adjusted to 150 bars; and
recovering spherical vitamin K nanoparticles after removal of the organic solvent (step 4).
